(19) United States
(12) Patent Application Publication
(10) Pub. No.: US 2024/0016888 A1
(43) Pub. Date: Jan. 18, 2024

(54) Wound Healing Promoting Protein

(71) Applicant: Eberhard Karls Universität Tuebingen Medizinische Fakultät, Tuebingen (DE)

(72) Inventor: Marius Ueffing, Tuebingen (DE)

(21) Appl. No.: 18/455,196

(22) Filed: Aug. 24, 2023

Related U.S. Application Data
(63) Continuation of application No. PCT/EP22/54637, filed on Feb. 24, 2022.

(30) Foreign Application Priority Data
Feb. 25, 2021 (DE) 102021104562.2

(51) Int. Cl. A61K 38/17

(52) U.S. Cl. CPC A61K 38/1709

(57) ABSTRACT

The present invention relates to a protein and polypeptide for use in promoting wound healing. It further relates to a nucleic acid molecule encoding the protein or polypeptide of the invention, an expression vector comprising said nucleic acid molecule, and a host cell comprising said expression vector.

### CROSS-REFERENCES TO RELATED APPLICATIONS

This application is a continuation of copending international patent application PCT/EP2022/054637 filed on 24 Feb. 2022 and designating the United States of America, which was not published under PCT Article 21(2) in English, and claims priority of German patent application DE 10 2021 104 562.2 filed on 25 Feb. 2021, all of which are herewith incorporated herein by reference in their entireties.

### FIELD OF THE INVENTION

The present invention relates to a protein and polypeptide for use in promoting wound healing. It further relates to a nucleic acid molecule encoding the protein or polypeptide of the invention, an expression vector comprising said nucleic acid molecule, and a host cell comprising said expression vector.

### BACKGROUND OF THE INVENTION

Wound healing is the closure of a wound due to the restoration of damaged body tissue by newly formed connective tissue in conjunction with epithelialization. It is a natural process that can be supported therapeutically. An injury in which wound healing is delayed or does not progress becomes a chronic wound.

Wound healing proceeds in different phases based on an interlocking of cells, such as fibroblasts, endothelia, immunocompetent cells, as well as cytokines, proteases and proteins of the extracellular matrix (ECM). Exactly how long each phase lasts can vary from individual to individual. In the repair of tissue damage, scars usually remain in the course of wound healing because the injured tissue is not specifically or completely replaced. Rather, non-specific connective and supportive tissue serves for this purpose to close the wound. In regeneration, wound healing takes place without scars, since the injured tissue is replaced quite specifically. Some tissues regenerate particularly well, such as skin and mucous membranes.

Various dispositions can interfere with or slow wound healing, for this purpose including wound infections, hypertension, diabetes mellitus, and disorders of connective tissue formation leading to pronounced, sometimes severely red, itchy scars. In addition, wound healing is often slowed down in old age.

Wound healing depends largely on the interaction between extracellular matrix and the cells involved in wound healing. Damaged or injured body tissue is thereby replaced by newly formed connective tissue due to epithelialization (overgrowth with epithelial cells). The various methods of wound treatment focus on the possibility of aseptic, rapid and scar-free closure and healing of the wound. Various methods of regenerative medicine (tissue engineering) aim to achieve wound closure even in cases where deep wounds, e.g., in the case of ulceration or traumatic injuries, do not ensure rapid wound closure, combined with the coordinated migration of cells and ECM formation into the wound. For this purpose, either autologous cells or those from foreign donors are used to produce skin equivalents. For example, keratinocytes, cells of the epidermis, are propagated in a three- to four-week process and subsequently moved to the wound directly or by means of a carrier. The duration of the subsequent healing process depends on the general conditions.

Despite multiple therapeutic methods for wound healing, the management of chronic, inflamed, or septically infected wounds remains a problem. Various systemic diseases such as diabetes delay wound healing. In hospitalized and especially elderly people, chronic wounds such as venous leg ulcer, pressure ulcer or diabetic foot ulcer lead to complications.

### SUMMARY OF THE INVENTION

Against this background, it is a problem underlying the invention to provide a novel therapeutically useful agent that is applicable for promoting wound healing.

This problem is solved due to the provision of age-related maculopathy susceptibility protein 2 (ARMS2) or an active fragment thereof for use in promoting wound healing.

In one embodiment, the provision according to the invention can be in the form of natural, native ARMS2 protein purified from biological material. However, it can also be in the form of a protein and/or polypeptide that can comprise the activity of natural native ARMS2, in particular the ability of ARMS2 to bind to extracellular matrix proteins. For this purpose, the protein and/or polypeptide and/or active fragment according to the invention contains exclusively the activity-bearing amino acid segments of ARMS2.

Against this background, the present invention is also synonymously representable by a protein and/or polypeptide for use in promoting wound healing, which comprises the activity of ARMS2. Therefore, the above and following properties, features and advantages apply equally to the protein and polypeptide according to the invention.

According to the invention, an “active fragment” is understood to be a polypeptide or protein that comprises the activity of natural, native ARMS2, in particular the ability of ARMS2 to bind to extracellular matrix proteins.

Age-related maculopathy susceptibility protein 2 (ARMS2) is a small secreted protein specifically found in primates. In its human variant, it is a protein with 107 amino acids and a size of 12 kD, whose coding sequence is located on chromosome 10q26.13 (Gene ID: 387715). The amino acid sequence (UniProtKB/Swiss-Prot: POC7Q2.1) and coding sequence (CCDS53585.1) of the human variant is indicated in the sequence listing under SEQ ID No. 1 and SEQ ID No. 2.

It is understood that the protein and/or polypeptide according to the invention can comprise the identical amino acids of the natural ARMS2 or the active fragment. Alternatively, however, the protein and/or polypeptide can comprise an amino acid sequence homologous thereto, wherein the ARMS2 activity does not change or does not change significantly. According to the invention, a homologous amino acid sequence means that at least one amino acid is exchanged for an amino acid of the same group.

It is known that the so-called proteinogenic amino acids can be divided into four groups, and that the exchange of an amino acid in a peptide for an amino acid of the same group often does not change the function of the peptide or changes it only slightly. Such an amino acid exchange can be useful in particular with regard to a chemical synthesis or a biotechnological production, if due to the exchange the corresponding peptide can be produced with a higher yield, wherein due to the exchange within one group the wound healing promoting effect is maintained.

For the sake of completeness only, it should be mentioned that the amino acid groups are characterized as follows: I. amino acids with neutral and hydrophobic (nonpolar) side chains, II. amino acids with neutral and hydrophilic (polar) side chains, III. amino acids with acidic and hydrophilic (polar) side chains, and IV. Amino acids with basic and hydrophilic (polar) side chains.

The ARMS2 and active fragment can be present unmodified according to the invention. Alternatively, they comprise at least one modification, such as glycosylation, alkylation, etc. Such modifications can serve the stability of the protein or polypeptide or an increased biological effectiveness, but can also be due to a certain form of biotechnological production. Furthermore, it can be useful to provide at least one of the amino acids of the peptide with a protective group to protect the peptide from attack by exopeptidases.

The protein or polypeptide according to the invention can preferably comprise L- and/or D-amino acids. According to the invention and generally understood, an “L-amino acid” refers to a stereoisomer of a particular amino acid whose amino group is on the left side in its Fisher projection, while “D-amino acid” refers to the other stereoisomer of the amino acid whose amino group is on the right side in its Fisher projection. While most naturally occurring peptides consist of amino acids in the L-configuration, D-amino acids have shown strong resistance to proteolytic degradation. Thus, according to a preferred embodiment, the protein or polypeptide of the invention can comprise D-amino acids. According to an alternative embodiment, the protein or polypeptide according to the invention comprises D-amino acids. According to a still further alternative embodiment, the protein or polypeptide according to the invention comprises a mixture of D- and L-amino acids.

The protein or polypeptide according to the invention can comprise N- and or C-terminal modifications. This makes it possible, for example, to influence or improve the stability or half-life of the protein or polypeptide according to the invention, in particular in environments that promote degradation and/or modification of the free N-/C-terminal ends of peptides, for example due to proteases present in these environments.

Examples of C-terminal modifications include -amide, -acide, -N-alkyl-amide, -aldehyde, -ester, -p-nitroanilide, and -7-amino-4-methylcoumarin. With these modifications, the C-terminal end of the protein or polypeptide can be protected without significantly affecting the activity of the peptide. Examples of N-terminal modifications include acetyl-, formyl-, pyroglutamyl-, fatty acid-, urea-, carbamate-, and alkylamine-. It is understood that either both ends, i.e., the N-terminus and the C-terminus, can be modified with one of the modifications described above, or only one of the ends, i.e., either the N- or the C-terminus.

According to the invention and common understanding, the C-terminus (also known as carboxyl-terminus, carboxy-terminus, C-terminal tail, C-terminal end or COOH-terminus) is the end of an amino acid chain (protein or polypeptide), terminated by a free carboxyl group (—COOH), and the N-terminus (also called amino terminus, NH₂ terminus, N-terminal end, or amine terminus) is the beginning of a protein or polypeptide and refers to the free amine group (—NH₂) located at the end of a polypeptide. When writing peptide sequences, it is common to put the C-terminal end on the right and write the sequence from the N- to the C-terminus.

The protein or polypeptide according to the invention can be produced recombinantly and/or purified from biological material. It can further be produced with methods of peptide synthesis. In the prior art, a variety of methods for the chemical synthesis of peptides are known. While chemical synthesis of peptides can be performed with classical solution-phase methods, these have been replaced by solid-phase methods in most research and development environments. An overview of peptide synthesis can be found, for example, in Stawikowski et al. (2012), Introduction to Peptide Synthesis, Cur. Prot. Sci., Suppl. 69, 18.1.1-18.1.13.

According to the invention, “promotion of wound healing” is understood to mean the targeted action on the wound closure process with the aim of improving and/or accelerating it compared to the natural process. Further encompassed according to the invention is the use in the treatment of wound healing disorders.

The inventors' findings were surprising. For example, in the prior art, the ARMS2 protein has so far been described in completely different contexts, namely in particular as a component of the choroidal extracellular matrix of the eye and its involvement in the development of eye diseases.

For example, Kortvely et al. (2010), AMRS2 Is a Constituent of the Extracellular Matrix Providing a Link between Familial and Sporadic Age-Related Macular Degenerations, Investigative Ophthalmology & Visual Science Vol. 1, No. 1, pp. 79- 88, describe that ARMS2 can play a role in the development of age-related macular degeneration (AMD). This is in particular true for genetic alterations of ARMS2; see Kanda et al. (2007), A Variant of Mitrochondrial Protein LOC387715/ARMS2, not HTRA1, is strongly associated with age-related macular degeneration, Proc. Natl. Acad. Sci. USA 104(41), pp. 16227-16232; Fritsche et al. (2008), Age-Related Macular Degeneration is Associated with Unstable ARMS2 (LOC387715) mRNA, Nat. Genet. 40(7), pp. 892-896. Further publications describing the importance of ARMS2 in AMD are: Biames et al. (2020), Genotype- and Phenotype-Based Subgroups in Geographic Atrophy Secondary to Age-Related Macular Degeneration: The EYE-RISK Consortium, Ophthalmol Retina 4(12), pp. 1129-1137; Nita et al. (2014), Age-Related Macular Degeneration and Changes in the Extracellular Matrix, Med. Sci. Monit. 20, pp. 1003-1016, and Vierkotten et al. (2011), Overexpression of HTRA1 Leads to Ultrastructural Changes in the Elastic Layer of Bruch's Membrane via Cleavage of Extracellular Matrix Components, PLoS One. 6(8), e22959.

The exact physiological function of ARMS2 has remained unclear.

Surprisingly, the inventors have recognized for the first time that ARMS2, in its interaction with the extracellular matrix (ECM), modifies it in such a way that it can assist wound healing during wound settlement. This includes endothelial cells in wound setting leading to vascular injury, as the inventors have evidence that ARMS2 is involved in adhesion and reorganization of endothelial cells after vascular wound setting. This was not expected in light of the prior art. It thereby engages in the natural wound healing process in a particularly advantageous way at the molecular level. Thus, it has been shown that the invention can improve and support the molecular ultrastructure of the ECM. According to the findings of the inventors, the protein and polypeptide according to the invention have a positive influence on the build-up and cross-linking of structure-forming collagen, fibulins and thus cross-linked elastin fibers as a component of a matrix for accelerated wound healing due to improved migration of cells into the area of the wound.

In one embodiment of the invention, human ARMS2 and/or the active fragment of human ARMS2 is preferably used.

This measure has the advantage of favoring direct application of the invention to humans, as it is believed that the human variant of ARMS2 exhibits particular efficacy in wound healing in humans.

In a further embodiment of the invention, the active fragment or protein and/or polypeptide of the invention comprises the activity of ARMS2 to bind extracellular matrix (ECM) proteins.

This measure has the advantage that not all of the full-length ARMS2 is used, but only the areas that are found by the inventors to play a particular role in wound healing activity. This has an advantageous effect on the production as well as the stability of the protein or polypeptide, the formulability into a drug and last but not least on the production costs.

In a further embodiment of the invention, the ECM proteins are selected from the group consisting of: Fibulin 6 (Hemicentin-1), Fibronectin-1, and Microfibril Interface Located Protein 2 (EMILIN2).

This measure has the advantage that the protein or polypeptide according to the invention is capable of establishing such ECM bonds, which are known by the inventors to play a particularly important role in promoting wound healing. In this embodiment, the protein or polypeptide is therefore optimized in its wound healing-promoting activity.

In a further embodiment of the invention, wound healing comprises healing of wounds of the skin.

This measure has the advantage that the protein or polypeptide according to the invention is configured for external application for the treatment of particularly important and frequently occurring wounds.

According to a further embodiment of the invention, wound healing comprises wound closure of blood vessels.

This measure has the advantage that with the invention, crucial and often deficient processes of vascular closure in hemorrhagic processes and wound healing can be engaged in a targeted manner. This includes enterohemorrhagic processes in internal organ bleeding.

According to a further embodiment of the invention, promoting wound healing promotes wound closure and/or scar formation.

This measure has the advantage that with the invention, crucial and often deficient processes of wound healing can be engaged in a targeted manner.

A further subject-matter of the present invention relates to a nucleic acid molecule encoding ARMS2 according to the invention or the active fragment thereof.

Once the amino acid sequence of the protein and polypeptide of the invention is known, a corresponding nucleic acid sequence can be derived using the genetic code, wherein optimized codons for different hosts (bacteria, yeast, mammalian cells) can be used. Furthermore, the coding nucleic acid sequence is known for a row of mammalian ARMS2, such as for the human variant: Gene ID: 387715.

The nucleic acid molecule of the invention can be DNA, RNA, including mRNA, which can be linear or circular, single or double stranded. The nucleic acid molecule can comprise natural or chemically modified nucleotides.

The features, properties, advantages and embodiments of the protein or polypeptide according to the invention apply correspondingly to the nucleic acid molecule according to the invention.

A further subject-matter of the invention relates to an expression vector comprising the nucleic acid molecule according to the invention and optionally nucleotide sequences for controlling replication, transcription and/or translation, such as, for example, promoter sequences, enzyme binding sequences, sequences for the origin of replication (ORI), etc.

A wide variety of expression vectors or systems can be used to produce the proteins or polypeptides of the invention. Such vectors include, but are not limited to, chromosomal, episomal, and virus-derived vectors, e.g., vectors derived from bacterial plasmids, from bacteriophages, from transposons, from yeast episomes, from insertion elements, from chromosomal yeast elements, from viruses, and vectors derived from combinations thereof, such as those derived from plasmid and bacteriophage gene elements, such as cosmids and phagemids. Expression system constructs can contain control regions that both regulate and elicit expression. In general, any system or vector capable of obtaining, propagating or expressing polynucleotides and/or expressing a polypeptide in a host can be used for expression in this context. The corresponding DNA sequence can be inserted into the expression system due to a variety of well-known and routine techniques, such as those described in Sambrook et al. (2012), Molecular Cloning: A Laboratory Manual: A Laboratory Manual, 4th edition, Cold Spring Harbor Laboratory Press.

A further subject-matter of the present invention also relates to a host cell comprising the expression vector of the invention.

As used herein, the term “host cell” is presently defined as a biological cell that has been transformed, transfected, or is capable of transformation or transfection due to an exogenous polynucleotide sequence encoding the protein or polypeptide of the invention.

The production of a protein or polypeptide according to the invention by nucleic acid expression has the advantage that the protein or polypeptide can be produced in virtually unlimited quantities. In addition, however, it can also be modified in a simple way, namely by modifying the corresponding coding sequence at the nucleic acid level so as to effect an amino acid exchange.

The features, properties, advantages and embodiments of the protein or polypeptide according to the invention apply correspondingly to the expression vector and host cell according to the invention.

In view of the above, the invention also relates to a pharmaceutical composition for promoting wound healing comprising ARMS2 according to the invention, or the active fragment thereof, or the nucleic acid molecule according to the invention, or the expression vector according to the invention, and a pharmaceutically acceptable carrier.

In general, a “pharmaceutically acceptable carrier” means any excipient, additive, or carrier typically used in the field of wound healing that facilitates or enables the administration of the composition of the invention to a living being and/or improves its stability and/or activity. The pharmaceutical composition can also include binders, diluents or lubricants. A pharmaceutical carrier or other additives can be selected based on the intended route of administration and standard pharmaceutical practice. Pharmaceutically acceptable carriers can include solvents, extenders, or other liquid binders such as dispersants or suspending agents, surfactants, isotonics, spreaders or emulsifiers, preservatives, encapsulants, solid binders, depending on what is most appropriate for the particular dosage regimen and also compatible with the compound of the invention.

Pharmaceutically acceptable carriers are well known in the prior art and are described, for example, in Rowe (2020), Handbook of Pharmaceutical Excipients, Pharmaceutical Press, 9th edition.

A further subject-matter of the present invention relates to a method for promoting wound healing, which comprises administering the protein and/or polypeptide and/or expression vector and/or pharmaceutical composition according to the invention to a living being, preferably a human being, and/or a wound.

The features, properties, advantages and embodiments of the protein or polypeptide according to the invention apply correspondingly to the method according to the invention.

It is understood that the features mentioned above and those still to be explained below can be used not only in the combinations indicated in each case, but also in other combinations or on their own, without leaving the scope of the present invention.

The invention will now be explained in more detail by means of examples. The features mentioned therein are considered to belong to the invention not only with respect to the specific example but also in isolated form.

Reference is thereby made to the accompanying figure.

### BRIEF DESCRIPTION OF THE FIGURE

FIG. 1 shows the result of a cellular “scratch assay” demonstrating that native ARMS2 helps to close a wounding of a cellular monolayer produced due to scratching, while mutant form 69S cannot and even hinders this.

### EXAMPLES

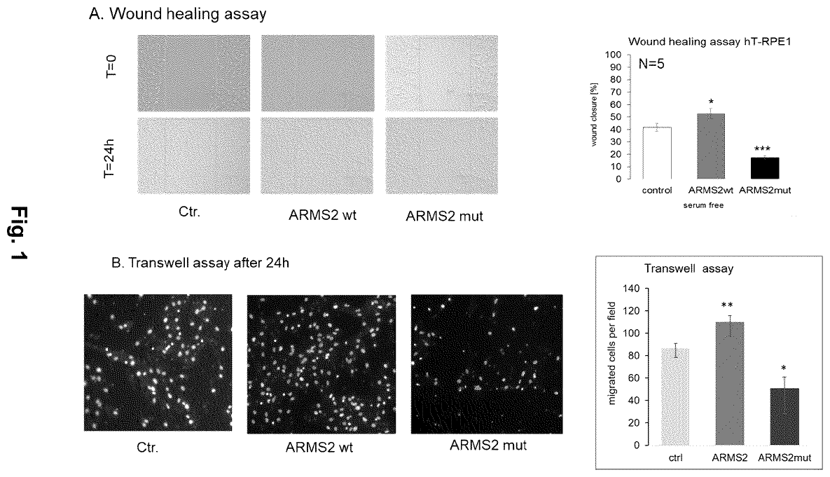

T-RPE1 cells were transfected with ARMS2 wild type (gray bars), the ARMS2 A69S mutant (dark gray bars) or empty vector (white bars). The result of the experiment is shown in FIG. 1.

A. Representative images of the scratches from the wound healing assay; right: wound closure after 24 h under serum-free conditions. * (t-test p<0.05) shows significantly increased cell migration of wild-type ARMS2 overexpressing cells compared to control. *** (t-test p<0.001) indicates significantly decreased cell migration of cells overexpressing the ARMS2 A69S mutant compared to control.

B. Transwell invasion assay, mean number of migrated cells per field. ** (t-test p<0.01), * (t-test p<0.05) show a significant difference compared to control. Representative images of transwell membranes stained with DAPI after 24 h.

The inventors were able to prove on the basis of a representative cell culture experiment that ARMS2 leads to a significant improvement in the wound healing process. In contrast, the use of an inactive ARMS2 mutant does not show the observed effects and even blocks the wound healing process. This experiment impressively demonstrates the therapeutic suitability of the protein and polypeptide of the invention for promoting wound healing.

### Sequences

SEQ ID NO:1: Amino acid sequence of human ARMS2 (full length).

SEQ ID NO:2: coding nucleotide sequence of human ARMS2 (full length).

1. A method for promoting wound healing, which comprises administering to a living being an age-related maculopathy susceptibility protein 2 (ARMS2) or an active fragment thereof.

2. The method of claim 1, wherein said ARMS2 is human ARMS2.

3. The method of claim 1, wherein the active fragment comprises the activity of ARMS2 to bind extracellular matrix (ECM) proteins.

4. The method of claim 3, wherein the ECM proteins are selected from the group consisting of: Fibulin 6 (Hemicentin-1), Fibronectin-1 and Microfibril Interface Located Protein 2 (EMILIN2).

5. The method of claim 1, wherein said wound healing comprises healing of wounds of the skin.

6. The method of claim 1, wherein said wound healing comprises wound closure of blood vessels.

7. The method of claim 1, wherein said promotion of wound healing promotes wound closure.

8. The method of claim 1, wherein said promotion of wound healing promotes scar formation.

9. The method of claim 1, wherein the living being is a human being.

10. A pharmaceutical composition for promoting wound healing comprising an age-related maculopathy susceptibility protein 2 (ARMS2) or an active fragment thereof, or a nucleic acid molecule encoding said ARMS2 or said active fragment thereof, or an expression vector comprising said nucleic acid molecule, and a pharmaceutically acceptable carrier.


SEQUENCE LISTING (2 sequences)

SEQ ID NO: 1 - protein, 107 aa, Homo sapiens
MLRLYPGPMVTEAEGKGGPEMASLSSSVVPVSFISTLRESVLDPGVGGEGASDKQRSKLSLSHSMIPAAKIHTELCLPAFFSPAGTQRRFQQPQHHLTLSIIHTAAR

SEQ ID NO: 2 - DNA, 324 bp, Homo sapiens
  source 1..324: mol_type = other DNA
atgctgcgcctatacccaggaccgatggtaactgaggcggaggggaaaggagggcctgagatggcaagtctgtcctcctcggtggttcctgtgtccttcatttccactctgcgagagtctgtgctggaccctggagttggtggagaaggagccagtgacaagcagaggagcaaactgtctttatcacactccatgatcccagctgctaaaatccacactgagctctgcttaccagccttcttctctcctgctggaacccagaggaggttccagcagcctcagcaccacctgacactgtctatcatccacactgcagcaaggtga